(19) United States
(12) Patent Application Publication
(10) Pub. No.: US 2011/0024647 A1
(43) Pub. Date: Feb. 3, 2011

(54) Ultraviolet toothbrush sanitizing-box

(76) Inventor: Chien-Chien Hsu, Burnaby (CA)

Correspondence Address: Hsu Chien-Chien, 4595 Portland Street, Burnaby, BC, V5J2P1, CA

(21) Appl. No.: 12/462,327

(22) Filed: Aug. 3, 2009

(51) Int. Cl. G01N 23/00

(52) U.S. Cl. 250/455.11

(57) ABSTRACT

The present invention provides an ultraviolet light toothbrush sanitizing-box for bacteria prevention and toothbrush dehumidification. The ultraviolet light toothbrush sanitizing-box will sterilize the toothbrush with ultraviolet light and dehumidify the toothbrush storage environment, thereby maintaining the toothbrush in a germ-free condition before each use and prolonging the life-span of the toothbrush.

### FIELD OF THE INVENTION

The present invention relates to a toothbrush container; more particularly, the present invention relates to a toothbrush container with automatic ultraviolet light sanitizing system.

### BACKGROUND OF THE INVENTION

Most people often brush their teeth in a rush, and the germs are left on the toothbrush in a wet condition after the use, leaving an ideal condition for germ-development, therefore, many report suggest that the toothbrush should be replaced every month or so; however, even replacing the toothbrush regularly can not guarantee that the toothbrush is always in the germ-free condition, as the germ development is fast in the bathroom if the food remains are carelessly stained on the toothbrush after the use.

Therefore, the main objective of the present invention is to provide a feasible solution to enhance the oral hygiene for everyone.

### SUMMARY OF THE INVENTION

1. It is the primary objective of the present invention to provide an ultraviolet toothbrush sanitizing-box capable of germ prevention and dehumidification to maintain the toothbrush in a germ-free condition before each use.

2. It is the second objective of the present invention to provide a safe and efficient control method of the toothbrush sanitizing-box, thereby preventing the user from directly facing the ultraviolet light during the toothbrush sanitizing process.

3. It is the third objective of the present invention to provide an ultraviolet toothbrush sanitizing-box for prolonging the life-span of the toothbrush.

### BRIEF DESCRIPTION OF THE DRAWINGS

FIG. 1 is the illustrative view of the ultraviolet toothbrush sanitizing-box.

FIG. 2 is another construction of the ultraviolet toothbrush sanitizing-box with a cylindrical shape.

### DESCRIPTION OF THE PREFERRED EMBODIMENTS

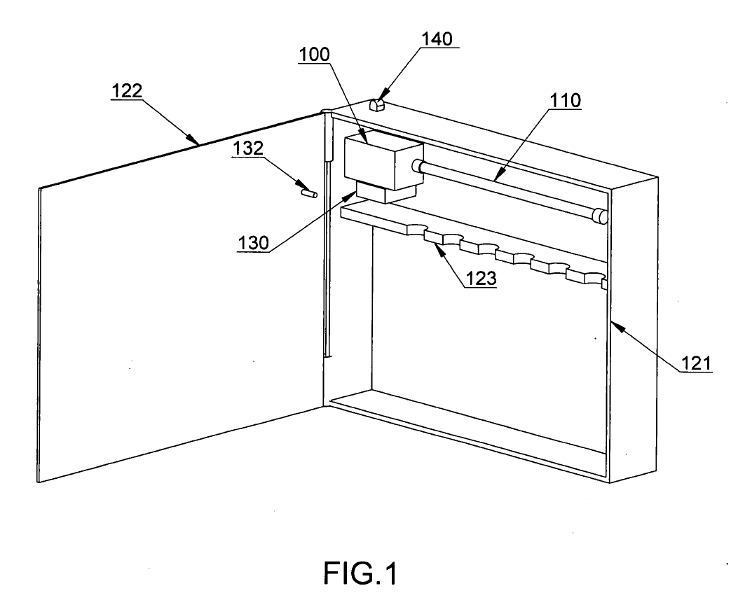

The present invention is shown in FIG.1, the components are labeled as follows, the toothbrush holder 123, the power-supply 100, the ultraviolet light tube 110, the lid member 122, the lid sensor 132, the sanitizing-box 121, the control circuit 130, the sanitizing process indication light 140.

The sanitizing-box 121 is preferable to be a a completely concealed space when the lid member 122 is shut, thereby keeping the water vapor out of the sanitizing-box 121 during the sanitizing process.

The lid member 122 will block the ultraviolet light from the ultraviolet light tube 110 during the sanitizing process; and at any given conditions, the control circuit 130 (or the lid sensor 132) will directly disable the power to the ultraviolet light tube 110 if the lid member 122 is detected to be in the open condition, thereby preventing the user from direct exposure to the ultraviolet light.

The sanitizing process indication light 140 will be turned on during the sanitizing process to signify the user that the sanitizing process is still ongoing.

There are two basic control methods for the present invention:

The first control method is defined as follows, when the control circuit 130 detects an opening action and a closing action of the lid member 122, the control circuit 130 will commence the sanitizing process by turning on the ultraviolet light tube 110 for a pre-set time period, the ultraviolet light will dehumidify and sterilize the toothbrush during this process.

The second control method is defined as follows, with the addition of a schedule-timer to the control circuit, the user can schedule one or more sanitizing processes at the certain time of the day with said schedule-timer, therefore, the user can make sure the toothbrush is in a germ-free condition prior to each use; this second method is more preferable in many common scenarios, for example, the user brush his teeth once at 11 pm and once at 6 am, even though the first control method can dehumidify and sterilize immediately after the use at 11 pm, there is still a time period of 7 hours for the germs to grow if the food remains are carelessly stained on the toothbrush during the storage, therefore, by setting the schedule timer to commence the sanitizing process at about 5:30 am and 10:30 pm in this scenario, the germ development on the toothbrush will be kept at a minimum level even if the toothbrush is not properly stored in a clean condition.

The best mode of the present invention is to embed both the first control method and the second control method in the control circuit, however, each method should be able to stand-alone for different user preference.

The power-supply of the ultraviolet toothbrush sanitizing-box can be a battery or a rechargeable battery.

The power-supply of the ultraviolet toothbrush sanitizing-box can be a power-supply directly connected to the wall plug.

The lid member and the sanitizing-box can further comprise mirror to reflect the ultraviolet light and increase the intensity of the ultraviolet light during the sanitizing process.

The sanitizing-box can further comprise a temperature sensor to detect the temperature change in the concealed space of the toothbrush during sanitizing process, if the temperature sensor detects an overheat condition, the control circuit will stop the sanitizing process.

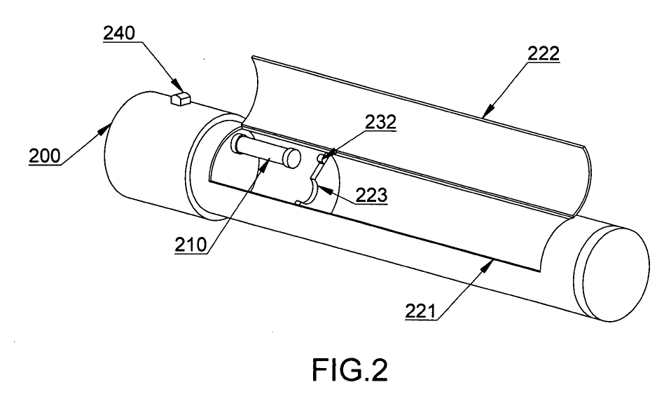

The sanitizing-box can be a cylindrical box as shown in FIG. 2, wherein all the basic components are similar to the component in FIG. 1 with a shape orientation to fit in a cylindrical sanitizing-box; the components are labeled as follows, the toothbrush holder 223, the power-supply 220, the ultraviolet light tube 210, the lid member 222, the lid sensor 232, the sanitizing-box 221, the control circuit (embedded with the power supply 220), the sanitizing process indication light 240.

1. An ultraviolet toothbrush sanitizing-box comprising:
a) a sanitizing-box 121;
b) a toothbrush holder 123 in said sanitizing-box 121;
c) a ultraviolet light tube 110 in said sanitizing-box 121 for commencing the sanitizing process;
d) a lid member 122 for concealing the ultraviolet light within said sanitizing-box 121;
e) a power-supply 100;
f) a lid sensor 132 for detecting the condition of the lid member 122;
g) a control circuit 130; wherein:
said control circuit will commence a sanitizing process by turning on said ultraviolet light tube 110 for a pre-set time period after a closing action of the lid member 122 is detected;
said control circuit will disable the power to the ultraviolet light tube 110 if the lid member 122 is detected to be in the open condition, thereby preventing the user from direct exposure to the ultraviolet light.

2. An ultraviolet toothbrush sanitizing-box as defined in claim 1, wherein; said sanitizing-box includes a sanitizing process indication light to signify when the ultraviolet light tube is turned on.

3. An ultraviolet toothbrush sanitizing-box as defined in claim 1, wherein; said sanitizing-box and said lid member can include mirror for intensify the ultraviolet light therein during the sanitizing process.

4. An ultraviolet toothbrush sanitizing-box as defined in claim 1, wherein; said sanitizing-box can include a temperature sensor to prevent the toothbrush from overheating.

5. An ultraviolet toothbrush sanitizing-box comprising:
a) a sanitizing-box;
b) a toothbrush holder in said sanitizing-box;
c) a ultraviolet light tube in said sanitizing-box for commencing the sanitizing process;
d) a lid member for concealing the ultraviolet light within said sanitizing-box;
e) a power-supply;
f) a lid sensor for detecting the condition of the lid member;
g) a control circuit with a schedule-timer; wherein:
said control circuit will commence a routine of the sanitizing process with ultraviolet light as scheduled by the user;
said control circuit will disable the power to the ultraviolet light tube if the lid member is detected to be in the open condition, thereby preventing the user from direct exposure to the ultraviolet light.

6. An ultraviolet toothbrush sanitizing-box as defined in claim 5, wherein; said sanitizing-box includes a sanitizing process indication light to signify when the ultraviolet light tube is turned on.

7. An ultraviolet toothbrush sanitizing-box as defined in claim 5, wherein; said sanitizing-box and said lid member can include mirror for intensify the ultraviolet light therein during the sanitizing process.

8. An ultraviolet toothbrush sanitizing-box as defined in claim 5, wherein; said sanitizing-box can include a temperature sensor to prevent the toothbrush from overheating.

9. An ultraviolet toothbrush sanitizing-box as defined in claim 5, wherein; said control circuit will commence a sanitizing process when an closing action of said lid member is detected.

10. An ultraviolet toothbrush sanitizing-box comprising:
a) a sanitizing-box;
b) a toothbrush holder in said sanitizing-box;
c) a ultraviolet light tube in said sanitizing-box for commencing the sanitizing process;
d) a lid member for concealing the ultraviolet light within said sanitizing-box;
e) a power-supply;
f) a lid sensor for detecting the condition of the lid member;
g) a control circuit and a schedule; wherein:
said control circuit will commence a sanitizing process by turning on said ultraviolet light tube for a pre-set time period after a closing action of the lid member is detected;
said control circuit will commence a routine of the sanitizing process with ultraviolet light as scheduled by the user;
said control circuit will disable the power to the ultraviolet light tube if the lid member is detected to be in the open condition, thereby preventing the user from direct exposure to the ultraviolet light.

11. An ultraviolet toothbrush sanitizing-box as defined in claim 10, wherein; said sanitizing-box includes a sanitizing process indication light to signify when the ultraviolet light tube is turned on.

12. An ultraviolet toothbrush sanitizing-box as defined in claim 10, wherein; said sanitizing-box and said lid member can include mirror for intensify the ultraviolet light therein during the sanitizing process.
